(19) United States
(12) Patent Application Publication
(10) Pub. No.: US 2026/0185135 A1
(43) Pub. Date: Jul. 2, 2026

(54) GALACTOOLIGOSACCHARIDE WITH HIGH LACTOSE CONTENT

(71) Applicant: DMK Deutsches Milchkontor GmbH, Zeven (DE)

(72) Inventors: Sven-Rainer DÖRING, Zeven (DE); Marco STEFFENS, Elsdorf (DE)

(21) Appl. No.: 19/422,875

(22) Filed: Dec. 17, 2025

(30) Foreign Application Priority Data
Dec. 19, 2024 (EP) 24221656.2

(51) Int. Cl. C12P 19/04; C12N 9/24; C12N 9/38; C12P 19/14

(52) U.S. Cl. CPC C12P 19/04; C12N 9/2402; C12N 9/2471; C12P 19/14; C12Y 302/01023; C12Y 302/01108

(57) ABSTRACT

Galactooligosaccharides characterized by a high lactulose content and a process for their production are proposed.

### AREA OF THE INVENTION

The invention relates to the field of food technology and concerns galactooligosaccharides characterized by a high lactulose content and a process for their production.

### TECHNOLOGICAL BACKGROUND

### Galactooligosaccharides (GOS)

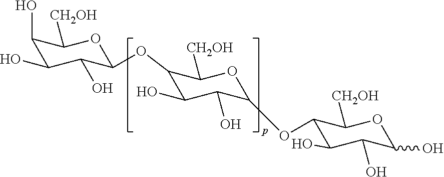

also known as oligogalactosyllactose, oligogalactose, oligolactose, or transgalactooligosaccharides (TOS), belong to the group of prebiotics. GOS are found in commercially available products such as food for infants and adults.

Due to the configuration of their glycosidic bonds, galactooligosaccharides (GOS) are largely resistant to hydrolysis by salivary and intestinal digestive enzymes. Galactooligosaccharides are therefore classified as prebiotics, defined as indigestible food components that have a beneficial effect on the host by stimulating the growth and/or activity of beneficial bacteria in the colon. The increased activity of these health-promoting bacteria leads to a number of effects, both directly through the bacteria themselves and indirectly through the organic acids they produce through fermentation. Examples of effects include stimulation of immune functions, absorption of essential nutrients, and synthesis of certain vitamins.

Galactooligosaccharides are a substrate for bacteria such as bifidobacteria and lactobacilli. Studies with infants and adults have shown that foods or beverages enriched with galacto-oligosaccharides lead to a significant increase in bifidobacteria. These sugars occur naturally in human milk and are known as human milk oligosaccharides. Examples include lacto-N-tetraose, lacto-N-notetraose, and lacto-N-fucopentaose.

The human gut microbiota plays a key role in the intestinal immune system. Galacto-oligosaccharides support the human body's natural defenses via the gut microflora, indirectly by increasing the number of bacteria in the gut and inhibiting the binding or survival of Escherichia coli, Salmonella Typhimurium, and Clostridia. GOS can positively influence the immune system indirectly through the production of antimicrobial substances by reducing the proliferation of pathogenic bacteria. Constipation is a potential problem, especially in infants, the elderly, and pregnant women. In infants, feeding with infant formula may be associated with constipation and hard stools. GOS can improve stool frequency and alleviate the symptoms associated with constipation.

### RELEVANT STATE OF THE ART

EP 2620506 B1 (DUPONT) relates to the production of GOS from lactitol.

EP 3598901 B1 (HOCHSCHULE ANHALT) relates to a method for producing GOS, in which a beta-galactosidase derived from L. bulgaricus (L. delbrueckii spp. bulgaricus) is incubated at a temperature of 37° C. or less with a lactose-containing composition such as milk, buffer, or whey, e.g., sweet whey, sour whey, whey concentrate, or whey permeate, at a temperature of 37° C. or less.

EP 3041945 B1 (FRIESLAND) provides a method for producing GOS from lactose, which (i) comprises contacting a lactose feed with immobilized beta-galactosidase (EC 3.2.1.23) and (ii) allowing GOS synthesis, wherein the lactose feedstock is an aqueous slurry of crystalline lactose.

WO 2008 037839 A1 (VALIO) relates to a method for producing GOS-containing milk-based products by treatment with a beta-galactosidase.

WO 2018 048305 A1 (UNIV GRONINGEN) describes the use of a GOS composition comprising branched and linear GOS species with a degree of polymerization (DP) of 3, wherein the branched DP3 GOS species are present in excess relative to the linear DP3 GOS species, for inducing mucin glycan utilization pathways in beneficial gut bacteria in an animal.

WO 2018 210820 A1 (NOVOZYMES) claims a method in which milk substrate with a lactose content of at least 20% by weight is treated with an enzyme having transgalactosylating activity. The transgalactosylating activity of the enzyme may have been increased by glycation of lysine and/or arginine residues through incubation of the enzyme with high glucose concentrations at elevated temperatures.

WO 2020 049016 A1 (FRIESLAND) relates to the field of hypoallergenic oligosaccharides for use in food compositions, in particular oligosaccharides with prebiotic properties. A hypoallergenic oligosaccharide composition is provided comprising galactooligosaccharides (GOS), wherein (i)) the content of galactooligosaccharides (GOS) is at least 40% by weight of the total dry matter of the composition; (ii) the content of allolactose is at least 10% by weight of the total dry matter of the composition; (iii) the content of 6′-GL is at least 30% by weight of the total GOS in the composition; and (iv) at least 0.5% by weight of the total GOS has a degree of polymerization (DP) of six or more.

WO 2020 117548 A1 (DUPONT) relates to a method for providing a low-lactose milk-based product with GOS fibers, wherein a milk substrate containing lactose is treated with a transgalactosylating enzyme to provide GOS fibers and residual lactose; deactivating the transgalactosylating enzyme; contacting the milk-based substrate with GOS fibers with a lactase to break down the remaining lactose to provide the low-lactose milk-based product with GOS fibers; and deactivating the lactase.

WO 2020 141032 A1 (FRIESLAND) relates to the field of food ingredients, in particular to economically attractive methods for producing hypoallergenic galactooligosaccharides (HA-GOS) and their use in food and feed. A method for producing an HA-GOS preparation is provided, which comprises bringing a lactose feedstock into contact with a specific beta-galactosidase (EC 3.2. 1.23), wherein the lactose feedstock is a cheese whey permeate (CWP) or a CWP enriched with sialyllactose (SL-CWP).

### OBJECT OF THE INVENTION

Lactulose is a well-known growth factor for Bifidobacterium. Its effectiveness in maintaining human health is well documented. Furthermore, the effectiveness of lactulose is not limited to humans, but has also been proven for animal feed, meaning that its application has been studied in a wide range of areas.

The effectiveness of lactulose in humans is reported, for example, in “The Many Faces of Lactulose: Recent Research Trends in Development and Physiological Effects” (Milk Science, Vol. 50, No. 2 (2001), pp. 39-47), which provides information on growth activity in Bifidobacterium. Through this activity, lactulose provides a range of effects, including improving the intestinal environment, improving excretion, and accelerating bowel movements, which means that its positive effects on human health are well known. Awareness of healthy eating has steadily increased among consumers in recent years. Taking probiotics is an important step for people who want to improve their overall health and well-being. Consequently, there is a need to provide consumers with new probiotics. The basic idea of the present invention is to provide a new probiotic composition containing two substances known for their probiotic effect.

The task of the present invention has therefore been to provide galactooligosaccharides with a high lactulose content. In addition to a high lactulose content, the galactooligosaccharides according to the invention should have a reduced amount of lactose and monosaccharides and low oligosaccharides.

### DESCRIPTION OF THE INVENTION

A first subject matter of the invention relates to galactooligosaccharides with a high lactulose content, obtainable or obtained by:
(a) providing an aqueous composition comprising lactose and fructose;
(b) sterilizing the aqueous composition from step (a);
(c) subjecting the lactose and fructose present in the sterilized aqueous composition from step (b) to transgalactosylation by adding at least one β-galactosidase within its optimal temperature and pH range for a period of at least 30 minutes to obtain a reaction mixture;
(d) inhibiting the enzyme mass contained in the reaction mixture from step (c);
(e) filtering the intermediate product from step (d) to obtain a retentate R1 comprising the inhibited enzymes and a permeate P1 comprising GOS, lactulose, lactose, and monosaccharides and low oligosaccharides;
(f) optionally treating the permeate P1 from step (e) with a lactose hydrolase and/or a yeast capable of metabolizing lactose to CO2 and ethanol, and then subjecting it to ultrafiltration to obtain a retentate R2 and a permeate P2;
(g) filtering the permeate P1 from step (e) or the permeate P2 from step (f) to obtain a retentate R3 and a permeate P3;
(h) packaging the retentate R3 from step (g).

A further subject matter of the invention relates to a process for the production of galactooligosaccharides, in particular galactooligosaccharides with a high lactulose content, comprising or consisting of the following steps:
(a) providing an aqueous composition comprising lactose and fructose;
(b) sterilizing the aqueous composition from step (a);
(c) transgalactosylating the lactose and fructose present in the sterilized aqueous composition from step (b) with the addition of at least one β-galactosidase within its optimal temperature and pH range for a period of at least 30 minutes to obtain a reaction mixture;
(d) inhibiting the enzyme mass in the reaction mixture from step (c);
(e) filtering the intermediate product from step (d) to obtain a retentate R1 comprising the inhibited enzymes and a permeate P1 comprising GOS, lactulose, lactose, and monosaccharides and low oligosaccharides;
(f) optionally treating the permeate P1 from step (e) with a lactose hydrolase and/or a yeast capable of metabolizing lactose to CO2 and ethanol, followed by ultrafiltration to obtain a retentate R2 and a permeate P2;
(g) filtration of the permeate P1 from step (e) or the permeate P2 from step (f) to obtain a retentate R3 and a permeate P3;
(h) packaging of the retentate R3 from step (g).

In a particular embodiment, the method according to the invention is characterized in that
(i) the enzyme mass is wholly or partially inhibited by setting a pH value outside the activity optimum; and
(ii) the inhibited enzyme quantity is separated as retentate (R1) and returned to step (c).

Surprisingly, it was found that galactooligosaccharide solutions can be produced by means of the method according to the invention, which have a lactulose content of at least about 5% by weight, whereby the amount of residual lactose, monosaccharides, and low oligosaccharides is below 10% by weight.

In accordance with the present invention, the lactulose content of the galactooligosaccharide mixtures according to the invention or the galactooligosaccharides produced by the method according to the invention is in the range of about 5 to 20% by weight, in particular about 10 to 15% by weight.

### Starting Materials

For the purposes of the present invention, aqueous compositions containing a sufficient amount of lactose, specifically glycosidically bound galactose, and fructose are suitable as feedstocks.

In a preferred embodiment, the aqueous composition is selected from the group consisting of fructose-enriched lactose solution, fructose-enriched sour whey, fructose-enriched milk permeates, or a syrup consisting of a mixture of glucose, galactose, and fructose.

In a further preferred embodiment, the above-mentioned aqueous compositions have a fructose content of about 10 to 25% by weight, in particular of about 15 to 25% by weight.

In a further preferred embodiment, the above-mentioned aqueous compositions have a lactose content of about 20 to 50% by weight, in particular of about 25 to 35% by weight.

In a further preferred embodiment, the above-mentioned aqueous compositions have a lactose content of about 20 to 50% by weight, in particular of about 25 to 35% by weight, and a fructose content of about 10 to 25% by weight, in particular of about 15 to 25% by weight.

When using the syrup, it is preferably prepared in advance from lactose. Preferably, pure lactose (pharmaceutical grade) is used. If lactose of lower quality is used, it can be purified in advance, for example by means of a cation exchanger to remove calcium. Using a β-galactosidase, the lactose is hydrolyzed to obtain a glucose-galactose syrup. This is followed by glucose isomerization to obtain a syrup consisting of glucose (25% by weight), galactose (50% by weight), and fructose (25% by weight). This process is well known to those skilled in the art and therefore does not need to be described in detail (see Luzzi, G., Steffens, M., Clawin-Rädecker, I., Hoffmann, W., Franz, C. M. A. P., Fritsche, J., and Lorenzen, P. C. (2020), Enhancing the sweetening power of lactose by enzymatic modification in the reformulation of dairy products. Int J Dairy Technol, 73:502-512). Of course, the fructose can be chromatographically isolated from the syrup, and this isolated fructose can be used to enrich an aqueous lactose solution or acid whey or a milk permeate with fructose.

It is advisable to use aqueous compositions in step (a) with a sufficiently high dry matter content to be able to carry out the method according to the invention with economically viable conversion rates and yields. Solutions with a dry matter content of about 25 to about 50 wt % and preferably about 30 to about 35 wt % are suitable for this purpose. If necessary, the aqueous compositions can be concentrated accordingly, for example by reverse osmosis (“RO”).

### Sterilization

The aqueous compositions from step (a) are sterilized. This refers to any process by which the germ load of the starting product can be reduced to a value below that specified by the respective national testing authorities as the threshold for approval as a foodstuff. As a rule, the aqueous compositions are sterilized to below 1,000 germs/mL, preferably to below 500 germs/mL and, in particular, to approximately 10 to approximately 50 germs/mL. The preferred sterilization method is high-temperature treatment, in which the aqueous compositions are exposed to a temperature in the range of about 70 to about 150° C., preferably about 90 to about 120° C., for about 3 to about 300 seconds, preferably about 50 to about 200 seconds.

### Enzymatic Transgalactosylation

The sterilized intermediates are subjected to enzymatic transgalactosylation in step (c). This refers to the transfer of galactose units to form an oligomeric sugar in the presence of suitable enzymes, in this case β-galactosidases, whereby enzymes from Aspergillus oryzae, Bacillus circulans, or mixtures of both are used together or in succession.

Aspergillus oryzae, more correctly Aspergillus flavus var. Oryzae, is a mold (watering can mold) that plays an important role in Japanese cuisine. It is the most important of the koji fungi. It is mainly used to ferment soybeans in solid-state bioreactors to produce miso and soy sauce.

Bacillus circulans is a species of bacteria that spreads circularly on culture media, which is where its name comes from. These are anaerobically growing, Gram-variable rod-shaped, motile cells that are 0.5 to 1 μm wide and 3.5 μm long. The bacterium ferments pentoses, hexoses, hexitols, and disaccharides. Bacillus circulans occurs in the intestines of herbivorous fish, where it aids digestion by excreting cellulases.

Like all enzymes, β-galactosidases also have a comparatively narrow temperature and pH range in which they perform optimally; these are well known to those skilled in the art.

With Aspergillus oryzae, the reaction is therefore preferably carried out at a temperature in the range of about 50 to about 60° C. and a pH value of about 4 to about 5, and, when enzymes from Bacillus circulans are added, at a temperature in the range of about 45 to about 55° C. and a pH value of about 5.5 to about 6.5. When Aspergillus oryzae is used, the term “acidic process” is therefore used, and when Bacillus circulans is used, the term “neutral process” is used.

A special feature of the basic formation of galactooligosaccharides is that the chain structure does not continue steadily, but loses speed after a while until even the competing reaction, namely the cleaving of the GOS, predominates.

It has therefore proven advantageous to take the enzyme-dependent reaction kinetics into account and to carry out the transgalactosylation over a period of approximately 30 to approximately 1200 minutes, and in particular from approximately 60 to approximately 90 minutes. It should be noted that high initial lactose concentrations significantly reduce the tendency to hydrolysis, and low enzyme concentrations also delay hydrolysis.

### Inhibition: pH Shift

Transgalactosylation is preferably carried out until the highest GOS concentration is reached. This value, which is determined by the enzyme and reaction conditions, can be monitored by sampling and is therefore easy for the specialist to determine. Once the maximum GOS formation has been reached, the activity of the enzymes must be stopped very quickly in order to prevent reverse cleavage. This can be done, for example, by rapid high-temperature heating, which, however, completely kills the enzyme material. The present invention prefers a different approach and removes the enzymes from their optimal reaction conditions, specifically by raising the pH value by at least two units above the optimum by adding bases or by lowering it by at least two units by adding acids. Although this pH shift does not bring the reaction to an abrupt halt, it reduces the activity of the enzymes by 80 to 90%, which is sufficient to prevent significant re-cleavage. To achieve this, it is sufficient to increase the pH to values of at least 7, preferably 8 to 12 and in particular 9 to 10, or to reduce it to values of 2 to 5, preferably 3 to 4.

When changing the pH value, it must be taken into account that the enzymes are not irreversibly inactivated and that the corresponding pH value-especially in the acidic range-does not prevent subsequent use of the product. The pH shift can be achieved by adding a required amount of common inorganic bases, such as aqueous NaOH, mineral acids such as HCl, or organic acids such as lactic acid. Raising the pH value is preferred over lowering it.

### Separation and Reuse of the Enzymes

The enzyme mass is preferably separated by filtration and, in particular, by ultrafiltration, which is also preferably carried out continuously. This produces a retentate R1 comprising the inhibited enzymes and a permeate P1 comprising GOS, lactulose, lactose, monosaccharides, and low oligosaccharides.

The separated enzyme quantity is returned to the reaction cycle in step (c). In the process, the enzymes are returned directly to the temperature and pH range in which they are at their optimum. If necessary, a fresh enzyme quantity can be added so that the enzyme activity remains constant or at least approximately constant throughout the continuous process.

As already mentioned, the enzyme mass is preferably separated by ultrafiltration. In a preferred embodiment, ultrafiltration is carried out at temperatures in the range of about 10 to about 55, preferably 10 to 20° C., with the membranes preferably having a pore diameter in the range of about 1 to about 50 kDa and preferably about 5 to about 25 kDa. Preferably, these are so-called spiral-wound membranes or plate-frame modules made of polysulfone or polyethylene membranes.

### Optional Post-Treatment

Optionally, the permeate P1 obtained in step (e) can be subjected to post-treatment by adding lactose hydrolase and/or a yeast that can metabolize the lactose residues to CO₂ and ethanol to the permeate P1.

Preferably, Kluyvermomyces lactis is used as the yeast. The post-treatment is also carried out within the optimal temperature and pH range of the hydrolase and/or yeast—usually between 28 and 37° C.—over a period of at least 30 minutes. Inhibition of the hydrolases and/or yeasts is not absolutely necessary in this case, but may be useful depending on the further intended use. This is followed by a second ultrafiltration step, which serves to separate the enzyme or enzyme/yeast mass. The result is a retentate R2, which contains enzyme or enzyme/yeast mass, and a permeate P2, which contains GOS and lactulose, among other things.

In a preferred embodiment, ultrafiltration is carried out at temperatures in the range of about 10 to about 55, preferably 10 to 20° C., wherein the membranes preferably have a pore diameter in the range of about 1 to about 50 kDa and preferably about 5 to about 25 kDa. Preferably, these are so-called spiral-wound membranes or plate-and-frame modules made of polysulfone or polyethylene membranes.

### 2. Filtration and Packaging (Purification, Concentration, and Drying)

The permeate P1 from step (e) or the permeate P2 from step (f) is subjected to filtration, yielding a retentate R3 containing GOS and lactulose and a permeate P3 containing undesirable by-products such as monosaccharides, lactose residues, and low oligosaccharides.

In a preferred embodiment, the filtration of step (g) is carried out by means of nanofiltration.

In a further preferred embodiment, nanofiltration is carried out at temperatures in the range of about 6 to about 60, preferably 6 to 20° C., wherein the membranes preferably have a pore diameter in the range of about 0.1 to about 2 kDa and preferably about 0.5 to about 1 kDa. Preferably, these are so-called spiral-wound membranes made of polymer materials or cartridge filters made of ceramic or aluminium oxide.

In order to obtain products that can be packaged, the retentate R3 is dried and, if necessary, purified and/or concentrated beforehand.

In order to increase the GOS concentration, the retentate R3 can be further purified, for example by electrodialysis or membrane processes such as reverse osmosis. If necessary, the dry mass can be increased by evaporation.

Drying is carried out, for example, by lyophilization, but preferably by spray drying, whereby the temperature at the inlet is typically about 180 to about 260° C. and at the outlet about 80 to about 105° C. The residual water content is a maximum of 5% by weight and preferably about 1 to about 2% by weight.

The present invention will be easier to understand with reference to the following examples. However, these examples serve only to illustrate the invention and cannot be interpreted as limiting the scope of protection of the invention.

### EXAMPLES

### Example 1

### Production of GOS by the Neutral Process Starting from a Fructose-Enriched Lactose Solution

1,000 kg of an aqueous lactose-fructose solution (30% lactose by weight, 18% fructose by weight) was heated to 98° C. in a tubular heat exchanger over 120 seconds and sterilized in the process. The sterilized solution was cooled to 55° C., transferred to a fermenter, adjusted to a pH value of 6.5 with lactic acid, mixed with β-galactosidase from Bacillus circulans in an enzyme: substrate weight ratio of 1:50, and stirred. The progress of transgalactosylation was monitored by sampling. The maximum GOS concentration was reached after approximately 90 minutes. The pH was increased to 10 within a few minutes by adding 30% by weight sodium hydroxide solution, which abruptly reduced the activity of the enzyme by 80%. The reaction mixture was fed to an ultrafiltration unit equipped with a spiral-wound membrane with a pore size of 10 kDa. The inactivated enzyme material was returned to the fermenter with the retentate R1; to compensate for losses, 5% by weight of fresh enzyme was added based on the initial amount. The permeate P1 was stirred with lactose hydrolase at an enzyme/yeast: substrate weight ratio of 1:25 and at 35° C. for about 10 hours. The reaction mixture was fed to a second ultrafiltration unit, which was also equipped with a spiral-wound membrane with a pore size of 10 kDa. The enzyme/yeast mass was separated with the retentate R2 and the permeate P2 was fed to a nanofiltration unit equipped with a ceramic membrane with a pore size of 500 to 1,000 Da, yielding a permeate P3 and a retentate R3. The monosaccharides still contained in the product were separated with the permeate P3, while the retentate R3 was fed to a reverse osmosis unit operating with a concentration factor of 1:2. The resulting permeate P4 (i.e., the concentration water) was returned to the process, and the retentate R4 (i.e., the GOS concentrate containing lactulose) was heated to approximately 85° C. in a plate heat exchanger for 30 seconds and sprayed over a tower. A white powder with a GOS content of more than 75% by weight and a lactulose content of 14% by weight was obtained, which still had a residual moisture content of 1% by weight and a monosaccharide content of 0.4% by weight.

What claimed is:

1. Galactooligosaccharides with a high lactulose content, obtainable or obtained by
(a) providing an aqueous composition comprising lactose and fructose;
(b) sterilizing the aqueous composition from step (a);
(c) subjecting the lactose and fructose present in the sterilized aqueous composition from step (b) to transgalactosylation by adding at least one β-galactosidase within its optimal temperature and pH range for a period of at least 30 minutes to obtain a reaction mixture;
(d) inhibiting the enzyme mass contained in the reaction mixture from step (c);
(e) filtering the intermediate product from step (d) to obtain a retentate R1 comprising the inhibited enzymes and a permeate P1 comprising GOS, lactulose, lactose, and monosaccharides and low oligosaccharides;
(f) optionally treating the permeate P1 from step (e) with a lactose hydrolase and/or a yeast capable of metabolizing lactose to CO2 and ethanol, and then subjecting it to ultrafiltration to obtain a retentate R2 and a permeate P2;
(g) filtering the permeate P1 from step (e) or the permeate P2 from step (f) to obtain a retentate R3 and a permeate P3;
(h) packaging the retentate R3 from step (g).

2. A process for producing galactooligosaccharides, in particular galactooligosaccharides with a high lactulose content, comprising or consisting of the following steps:
(a) providing an aqueous composition comprising lactose and fructose;
(b) sterilizing the aqueous composition from step (a);
(c) transgalactosylating the lactose and fructose present in the sterilized aqueous composition from step (b) by adding at least one β-galactosidase within its optimal temperature and pH range over a period of at least 30 minutes to obtain a reaction mixture;
(d) inhibiting the enzyme mass in the reaction mixture from step (c);
(e) filtering the intermediate product from step (d) to obtain a retentate R1 comprising the inhibited enzymes and a permeate P1 comprising GOS, lactulose, lactose, and monosaccharides and low oligosaccharides;
(f) optionally treating the permeate P1 from step (e) with a lactose hydrolase and/or a yeast capable of metabolizing lactose to CO2 and ethanol, followed by ultrafiltration to obtain a retentate R2 and a permeate P2;
(g) filtration of the permeate P1 from step (e) or the permeate P2 from step (f) to obtain a retentate R3 and a permeate P3;
(h) packaging of the retentate R3 from step (g).

3. Method according to claim 2, characterized in that: (i) the enzyme mass is wholly or partially inhibited by setting a pH value outside the activity optimum; and (ii) the inhibited enzyme quantity is separated as retentate (R1) and returned to step (c).

4. The process according to claim 2, wherein the aqueous composition in step (a) is selected from the group consisting of fructose-enriched lactose solution, fructose-enriched acid whey, fructose-enriched milk permeates, or a syrup consisting of a mixture of glucose, galactose, and fructose.

5. The process according to claim 2, wherein in step (a), aqueous compositions having a dry matter content of about 25 to about 50% by weight are used.

6. The process according to claim 2, wherein sterilization is effected by high-temperature treatment.

7. The process according to claim 2, wherein enzymes from Aspergillus oryzae and/or Bacillus circulans are used as β-galactosidase.

8. The process according to claim 7, wherein transgalactosylation is carried out by adding β-galactosidase from Aspergillus oryzae at a temperature in the range of about 50 to about 60° C. and a pH value of about 4 to about 5.

9. The process according to claim 7, wherein transgalactosylation is carried out by adding β-galactosidase from Bacillus circulans at a temperature in the range of about 45 to about 55° C. and a pH value of about 5.5 to about 6.5.

10. The process according to claim 2, wherein transgalactosylation in step (c) is carried out over a period of about 30 to about 1200 min.

11. The process according to claim 2, wherein the filtration in step (e) is ultrafiltration.

12. The process according to claim 2, wherein the post-treatment is carried out within the optimum temperature and pH value range of the hydrolase and/or yeast over a period of at least 30 minutes.

13. The process according to claim 2, wherein the filtration in step (g) is nanofiltration.

14. The process according to claim 2, wherein the retentate R3 is concentrated and dewatered.
